(19) United States
(12) Patent Application Publication
(10) Pub. No.: US 2025/0017761 A1
(43) Pub. Date: Jan. 16, 2025

(54) BODY URINAL FOR MALE PATIENT

(71) Applicant: MEDIRUNS CO., LTD., Bucheon-si (KR)

(72) Inventor: Bong Joo LEE, Bucheon-si (KR)

(21) Appl. No.: 18/706,808

(22) PCT Filed: Dec. 9, 2021
(86) PCT No.: PCT/KR2021/018564

§ 371 Date: May 2, 2024

(30) Foreign Application Priority Data
Dec. 7, 2021 (KR) 10-2021-0174113

(51) Int. Cl. A61F 5/453; A61B 5/20; A61B 10/00; A61F 5/441; A61L 2/08; A61L 2/26

(52) U.S. Cl. CPC A61F 5/453; A61B 5/20; A61B 10/007; A61F 5/441; A61L 2/085; A61L 2/26

(57) ABSTRACT

The present invention relates to a body-worn urinal for male patients, which is easy to wear even when the patient is lying down or moving, and which is capable of ventilation even when being worn for a long time without causing discomfort due to a foreign body sensation or pain, and is capable of sterilization and treatment by means of a built-in infrared sterilizer, thereby enabling natural urination and hygienic maintenance. Disclosed is the body-worn urinal for male patients, comprising: a body connecting and fixing device formed of a holder for fixing male genitalia; a urine collection cup for collecting urine, the urine collection cup comprising a water collection part formed on the upper side thereof and having a spherical inner space, a lamp holder formed on the upper end of one side of the water collection part so as to penetrate therethrough, a connection part which is formed on the other side of the water collection part and is open such that the body connecting and fixing device is connected thereto, and a drainage part which is formed downward from the water collection part so as to discharge urine; a far-infrared sterilizer coupled to the lamp holder of the urine collection cup; a urine discharge hose for discharging the urine collected in the urine collection cup; and a urine container to which the urine discharge hose is connected so as to temporarily store the urine, wherein the body connecting and fixing device further comprises Velcro attachment bands which are connected and fixed to both side ends of the outside thereof so as to be attached and fixed to Velcro attachment portions of a patient diaper.

### TECHNICAL FIELD

The present invention relates to a body-worn urinal for male patients, and more specifically, to a body-worn urinal for male patients, which is easy to wear even when the patient is lying down or moving, and which is capable of ventilation even when being worn for a long time without causing discomfort due to a foreign body sensation or pain, and is capable of sterilization and treatment by means of a built-in infrared sterilizer, thereby enabling natural urination and hygienic maintenance.

### BACKGROUND ART

Generally, elderly patients, trauma patients, wheelchair patients, surgical patients, and patients with urinary incontinence cannot urinate on their own.

In particular, patients classified as seriously ill patients in hospitals are unable to do anything on their own, and therefore always need the help of others.

Also, patients who can use their hands are not classified as seriously ill patients, but if the behavior is not possible, they can hardly do on their own.

For these patients, handling urine and feces is important in caring for the patients as much as eating.

Diapers are the most commonly used for this purpose.

Diapers cause harmful effects such as rashes and the like when worn for a long period of time, and patients cannot know exactly the time point when replacing diapers, which can lead to uremic toxin and make it more difficult for the patients.

In addition, since diapers are for one-time use, there is an uneconomical disadvantage in that several diapers must be used even a day for urinary treatment that is performed several times a day.

Thus, hospitals have recently recommended using a separate body urinal to ensure that urine is disposed of in a sanitary manner.

As the most commonly used body urinals for urine disposal, Korean Registered Patent No. 10-0940031 entitled “Body urinal for patients” discloses that the body urinal not only simply prevents noise during the inflow of urine and prevents the smelly urine from flowing back into the interior, but also can only be used when the patients indicate their intention to urinate or attempt to dispose of the urine themselves, which has limitations that it cannot be used for unconscious or sleeping patients.

Among the body urinals proposed to solve these limitations, Korean Registered Utility Model No. 20-0461172 entitled “Urine collection device for male patients” discloses that an urine collection device is constructed such that an penile insert and an urine collection container are coupled to each other through a hole in the underwear pants, and the urine collection device is made openable and closable by the lid of the container, whereby the penis is inserted through the penile insert while wearing panties, and when urine is discharged, it is collected in a urine collection container and discharged to the outside via a urine discharge hose on one side, and collected in a urine collection tube on the outside.

However, in the above-described prior arts, in order to clean the penile insert that is contaminated with urine during urination, the container for collecting urine, and the lid of the container, there was the inconvenience of having to take off the panties or separate these components from the pants every time washing, and there was also a problem that the maintenance is difficult.

Therefore, the present applicant has recently proposed the Korean Registered Patent No. 10-2224322 entitled “Body-worn urinal for male patients,” which was previously filed and registered, but there are still deficiencies in patient comfort and hygiene. Therefore, there is a need to improve a body-worn urinal for male patients that is not only stable and comfortable for the patient to wear, but also hygienic.

### DETAILED DESCRIPTION OF THE INVENTION

### Technical Problem

Therefore, the present invention was designed to solve the conventional problems as described above, and an object of the present invention is to provide a body-worn urinal for male patients that not only forms a fixing device wrapping around the genitals of the body in a divided manner to enable stretchable adjustment regardless of the size of the penis, but also enables stable fixation and wearing of a sanitary diaper using a fixing band. Another object of the present invention is to provide a body-worn urinal for male patients that fixes the genitals of the body and installs a far-infrared lamp in the place where urine is collected to enable disinfection and treatment.

Another object of the present invention is to provide a body-worn urinal for male patients that provides a diagnostic test kit to a urine collection container that stores urine to check for hematuria, proteinuria, diabetes, etc. and confirm the health condition.

Yet another object of the present invention is to provide a body-worn urinal for male patients that can be immediately replaced in the shape of a urine collection container or a urine bag depending on the patient's condition that are lying down or moving in the structure of one body genital fixing device.

### Technical Solution

In order to achieve the above object, according to the present invention, there is provided a body-worn urinal for male patients comprising: a body connecting and fixing device formed of a holder for fixing male genitalia; a urine collection cup for collecting urine, the urine collection cup comprising a water collection part formed on the upper side thereof and having a spherical inner space, a lamp holder formed on the upper end of one side of the water collection part so as to penetrate therethrough, a connection part which is formed on the other side of the water collection part and is open such that the body connecting and fixing device is connected thereto, and a drainage part which is formed downward from the water collection part so as to discharge urine; a far-infrared sterilizer coupled to the lamp holder of the urine collection cup; a urine discharge hose for discharging the urine collected in the urine collection cup; and a urine container to which the urine discharge hose is connected so as to temporarily store the urine, wherein the body connecting and fixing device further comprises Velcro attachment bands which are connected and fixed to both side ends of the outside thereof so as to be attached and fixed to Velcro attachment portions of a patient diaper.

Also, the body connecting and fixing device comprises: a connection coupling for which one end of the outer periphery is detachably coupled to the connecting part of the urine collection cup; and a divided packing cap which consists of a stretchable peripheral ring separated into a plurality of numbers while protruding and extending with a certain diameter and length to the center along the inner peripheral edge of the connection coupling, and which is formed to fit the body's genitals closely.

In addition, a protruding ring part is further formed to protrude on both sides of the outer periphery of the connection coupling, and connect and fixes a Velcro attachment band.

Further, the far-infrared sterilizer may comprise an infrared lamp that is coupled to a lamp holder having a hole formed at the upper end of one side of the urine collection cup, and is connected to the lamp holder by a socket and a cable.

Further, the urine container may comprise a diagnostic test kit consisting of a urine test bottle and a urine test stick that are compartmentalized and formed on one side and allow urine flowing in from the urine discharge hose to be tested.

Further, the Velcro attachment band of the body connecting and fixing device may be attached and fixed to the Velcro attachment portion of patient diaper pants worn by the patient.

Further, the patient diaper pants may comprise a back plate band formed to protrude on both sides and wrap around the back plate having both left and right wings having a Velcro structure; and a front plate shielding band that is formed to have a constant width in the front while being connected to a back plate band and one connecting band, thereby covering the patient's genitals, wherein the front plate shielding band is configured such that its front surface portion is composed of a Velcro attachment portion, and its center is formed with a hole that connects the patient's genitalia.

The body-worn urinal for male patients may comprise a bag fixing holder which is configured to have a connecting locking jaw formed to protrude outward with a certain width at the lower end portion of the drainage part of the urine collection cup, and which forms a urine container removably attached to the drainage part of the urine collection cup, and is coupled and fixed to the connection locking jaw; a bag fixing cap on which the bag is inserted into the fixing holder on the upper side and which is fastened with screws; a urine plastic bag formed on the lower side to temporarily store urine; a bag guiding bag that puts into a urine plastic bag and fixes it to the leg during walking or rehabilitation exercise; a plurality of leg fixing bands that is connected to the upper, middle and lower ends of the bag guiding bag and fixed with Velcro; and a body urinal position fixing band that is connected to the uppermost end of the bag guiding bag, and attached and fixed to the Velcro attachment portion of the patient diaper pants.

Further, the drainage part of the urine collection cup has a nipple structure, which is configured to be connected to a urine plastic bag and a straw-structure urine drain pipe to drain urine.

In addition, a diagnostic test kit is provided on one side of the urine container, wherein the diagnostic test kit consists of a urine test bottle that is inserted and mounted on one side of the upper end of the body of the urine container; and a urine test stick that is inserted into the inside of a urine test bottle to apply urine.

### Advantageous Effects

The present invention can provide a body-worn urinal for male patients which is formed so as to divide a fixing device wrapping around the genitals of the body, and thus can be adjusted flexibly regardless of the size of the penis, and enables stable fixation and wearing of a sanitary diaper using a fixing band.

Also, the present invention has the effect of fixing the body's genitals and installing a far-infrared lamp in the place where urine is collected, thereby enabling disinfection and treatment.

In addition, the present invention has the effect of providing a diagnostic test kit to a urine collection container that stores urine, and checking for hematuria, proteinuria, diabetes, and the like to confirm the health condition.

Moreover, the present invention can provide a body-worn urinal for male patients that can be easily replaced in the form of a urine collection container or a urine bag depending on the state of the patient where the patient is lying down or moving in the structure of a body genital fixing device.

In other words, trauma patients who are being treated for dementia, paralysis, cancer, and the like while lying down is stripped of their diapers, put on AeroCool diaper pants, and then wear a far-infrared body urinal to collect and process urine while lying down, and then use the built-in low-temperature device to process it, and disinfect and treat with the built-in low-temperature infrared sterilizer for 10 to 20 minutes. In addition, it is a type of medical device that can check the patient's health condition by checking hematuria, proteinuria, diabetes, and the like using a diagnostic stick built into the urinal container, and infrared disinfection and treatment can be performed 1˜2 times/day depending on the patient's condition.

The present invention can provide an environmentally friendly medical device that eliminates a current difficult task that a patient's guardian comes face-to-face with the patient and replaces diapers 4-5 times a day, reduces discomfort, odor, leakage, and waste caused by diapers, allows low-temperature disinfection treatment to be performed once or twice a day, and improves urinary tract infections, inflammation, bedsores, and pain.

### BRIEF DESCRIPTION OF THE DRAWINGS

FIGS. 1 to 6 are embodiments showing a body-worn urinal for male patients according to the present invention, in which:

FIG. 1 is a diagram illustrating the use of a body-worn urinal for male patients according to the present invention in a mounted state;

FIG. 2 is an enlarged illustrative view of the main part of FIG. 1;

FIG. 3 is a diagram illustrating the use of a body-worn urinal for male patients according to the present invention in a worn state;

FIG. 4 is an enlarged illustrative view of the main part of FIG. 3;

FIG. 5 is an exemplary diagram showing a state in which the body-worn urinal for male patients according to the present invention is worn; and

FIG. 6 is an exemplary view showing an adult diaper and its wearing used in combination with a body-worn urinal for male patients according to the present invention.

### DETAILED DESCRIPTION OF THE EMBODIMENTS

While the present invention may be modified in various ways and take on various alternative forms, specific embodiments thereof are shown in the drawings and described in detail below. However, it should be understood that there is no intent to limit the present invention to the particular forms disclosed, but on the contrary, the present invention covers all modifications, equivalents, and alternatives falling within the spirit and scope of the present invention.

The present invention is not limited to the embodiments disclosed below, but may be implemented in various forms different from each other, and the present embodiment is provided so that this disclosure will be thorough and complete, and will fully convey the scope of the invention to those skilled in the art.

Hereinafter, preferred embodiments of the present invention will be described in detail with reference to the accompanying drawings. Throughout this specification, reference should be made to the drawings, in which the same reference numerals and symbols will be used to designate the same or like components. In the following description of the present invention, detailed descriptions of known functions and components incorporated herein will be omitted in the case that the subject matter of the present disclosure may be rendered unclear thereby.

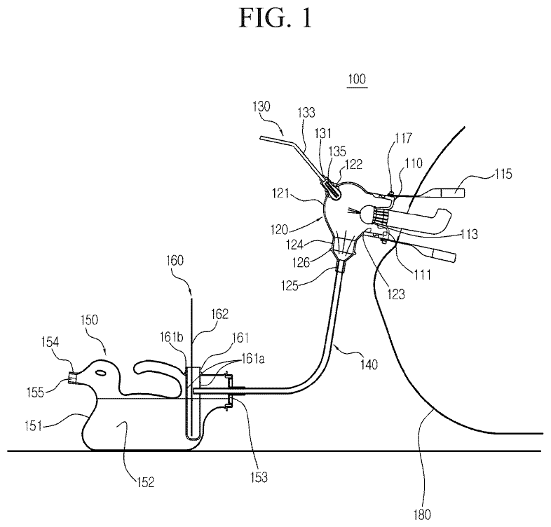

FIGS. 1 to 5 are embodiments showing a body-worn urinal for male patients according to the present invention.

Referring to FIGS. 1 to 4, the body urinal 100 for male patients according to the present invention comprises a body connecting and fixing device 110 formed of a holder for fixing male genitalia; a urine collection cup 120 which is configured so that the body connecting and fixing device 110 is connected thereto, and which collects urine, a far-infrared sterilizer 130 coupled and mounted to the urine collection cup 120; a urine discharge hose 140 for discharging the urine collected in the urine collection cup 120; and a urine container 150 to which the urine discharge hose 140 is connected so as to temporarily store the urine.

Further, the body connecting and fixing device 110 may further comprise Velcro attachment bands 115 which are connected and fixed to both side ends of the outside thereof so as to be attached and fixed to Velcro attachment portions of a patient diaper.

The body connecting and fixing device 110 may comprise a connection coupling 111 for which one end of the outer periphery is detachably coupled to the connecting part of the urine collection cup 120; a divided packing cap 113 which consists of a stretchable peripheral ring separated into a plurality of numbers while protruding and extending with a certain diameter and length to the center along the inner peripheral edge of the connection coupling 111, and which is formed to closely fit to the genitals of the body; and a Velcro attachment band 115 is connected and mounted to both ends of the connection coupling 111 and coupled to the Velcro attachment portion.

Further, a protruding ring part 117 may be further formed to protrude on both sides of the outer periphery of the connection coupling 111, and connect and fixes a Velcro attachment band 117.

Preferably, the body connecting device 110 is made of silicone, and can be formed to have stretchability depending on the size of the genital inserted into the divided packing cup 113, which is formed so that the tubular main surface extending from the connection coupling 111 to the center is cut into a plurality of pieces each having a certain length and a certain width from an end formed in the direction of the urine collection cup 120.

In addition, the divided packing cup 113 is formed in a shape in which the outer peripheral surface of a certain length from the tip is reduced to have a smaller diameter, so that the area with a reduced diameter can be formed to fit more closely to the penis at the glans border of the penis. In particular, the outer peripheral surface of the divided packing cup 113 may be formed to have a thickness that gradually decreases from a certain length toward the distal end, so that the penis can be inserted more smoothly while stretching in the longitudinal direction when the penis is inserted, and at the same time, may be formed to further improve adhesion to the penis.

That is, the divided packing cup 113 has its tip formed in a trumpet shape whose outer diameter widens in the direction of the urine collection cup 120, so that the tip of the divided packing cup 113 is in close contact with the rear surface of the glans, thereby further increasing adhesion to the penis. Thereby, the degree of tightening can be maintained at a level that does not cause any pain to the penis.

The urine collection cup 120 may comprise an upper water collection part 121 having a spherical inner space, a lamp holder 122 formed through an upper end of one side of the water collection part, a connection part 123 formed to be open so that the body connecting device is connected to the other side, and a drainage part 124 formed downward from the water collection part to discharge urine.

Preferably, the water collection part 121 has a spherical shape whose interior is empty, wherein the empty interior can be formed sot that urine jetted from the genitals can be collected. One side of the spherical water collection part 121 is formed with a connection part 123 by opening with a certain diameter and length on the outside, wherein such a water collection part 121 is opened downward with a certain diameter and length, thereby forming a drainage part 124 that discharges urine, and the lower part of the drainage part 124 may be formed with a nipple 125 that extends and protrudes downwardly to a certain length with a smaller diameter.

Here, the lamp holder can provide a structure which remove the lamp through a detachment/attachment method that allows the lamp to be mounted and then dismantled only during infrared sterilization, and which acts as a ventilation for air to pass through the bottom hole of the holder when disinfection is not performed. When the patient lies on the left or right side, the urine collection cup can be rotated left or right by the connection part. Even if the patient lies on the side too much and urine accumulates towards the lamp holder, water leakage occurs only when exceeding the height of the hole in the lamp holder, which makes it difficult for leakage to occur.

Meanwhile, the lower end of the drainage part 124 is configured to form a connection hooking jaw 126 that protrudes to have a certain width as the outer diameter, so that the upper end of the directly connected urine container or urine bag is formed to wrap around the outer peripheral surface of the drainage unit 124, thereby fixing the upper end of the urine collection bag using a fixing means such as a Velcro band or a rubber band.

In addition, the water collection part 121 may further form a lamp holder 123 that is fitted into a portion formed through an upper end of one side. This lamp holder 123 may be equipped with a far-infrared sterilizer 130 having an infrared lamp 135 connected to the socket 131 and a cable 133. As a result, it is possible to collect and process urine while lying down using a far-infrared sterilizer, and then disinfect and treat with the built-in low-temperature infrared sterilizer for 10 to 20 minutes.

The urine discharge hose 140 is a moving passage that discharges urine into the urine container 150, and it is more desirable to form of a material that is easy to bend and deform and has good restoring force even when bent.

The urine container 150 includes a body 151, a urine storage space 152 that can store a certain amount of urine inside, a drain cap 153 that is fixed by fitting coupling so that the other end of the urine discharge hose 140 is connected on one side of the body 151, and a discharge port 154 that allows urine filled inside to be discharged to the outside on the other side of the body 151, wherein the discharge port 154 can be formed to be opened and closed by a drainage air hole 155.

Meanwhile, one side of the urine container 150 can be configured to be equipped with a diagnostic test kit 160.

The diagnostic test kit 160 may comprise a urine test bottle 161 inserted through and mounted on one side of the upper end of the body 151 of the urine container 150, and a urine test stick 162 that is inserted into the inside of the urine test bottle 161 and is provided to apply urine. At this time, the urine test bottle 161 may be formed with through holes 161a on both sides of the upper end, whereby both sides are opened to allow urine to flow, a urine test stick can be dipped into the urine test stick to perform tests, and the urine test bottle can be withdrawn out from the urine container, put in a transport collection bottle, and sent to a pathology room. In addition, the urine test bottle is formed with a circumference jaw 161b at the upper part, so that it is inserted into the upper insertion part of a duck-shaped urine container to prevent it from falling down, and is opened on both front and back sides to allow urine to flow, and a urine hose is inserted on one side. It is possible to open the upper lid, store the test stick, and perform a test. Alternatively, it is possible to withdraw out the test bottle, put in a collection bottle, and send to the pathology room to perform a detailed test.

On the other hand, it is more preferable that the urine container 150 has a size that can sufficiently store at least one dose of urine, and a urine storage space 152 is formed, and the bottom surface is formed with a flat surface of a sufficient size to enable stable landing anywhere. For this purpose, the urinal container 150 may be formed in the shape of various more familiar animals or livestock, including a duck shape as shown in the illustration, but is not limited thereto.

Therefore, the urine container 150 can be formed close to the patient or at a farther distance depending on the length of the urine discharge hose 140.

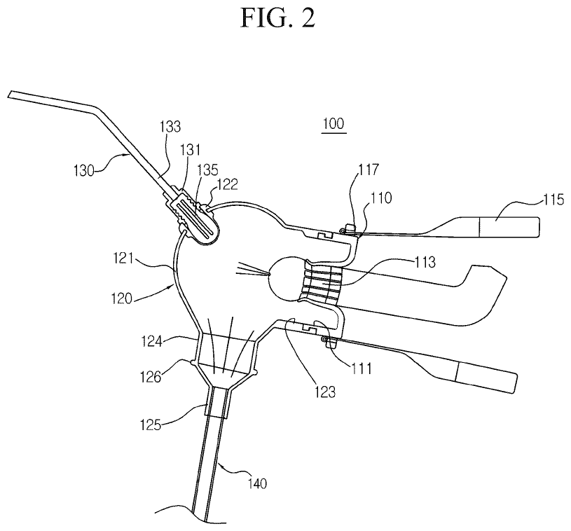

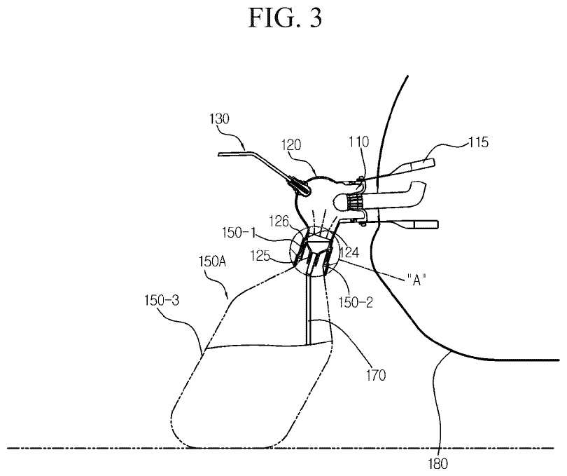

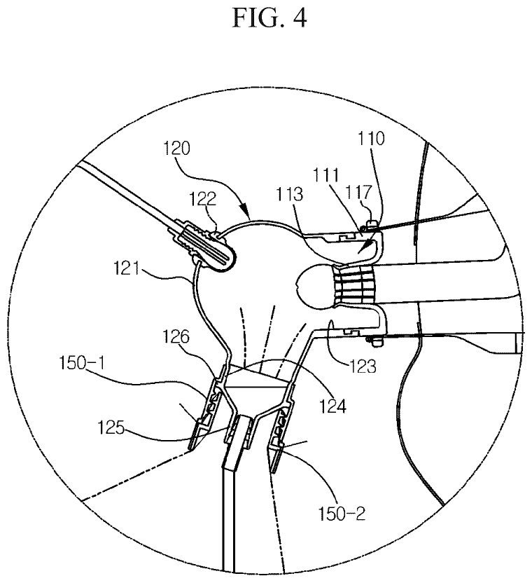

FIG. 2 is a diagram illustrating the use of the body-worn urinal for male patients according to the present invention in a worn state; FIG. 3 is an enlarged illustrative view of the main part of the “A” part of FIG. 2, and FIG. 4 is an exemplary diagram showing a state in which the body-worn urinal for male patients according to the present invention is worn.

Referring to FIGS. 2 to 4, the body-worn urinal for male patient may comprise a bag fixing holder 150-1 which is configured to have a connecting locking jaw 126 formed to protrude outward with a certain width at the lower end portion of the drainage part 124 of the urine collection cup 120, and which forms a urine container 150A, and is coupled and fixed corresponding to the connection locking jaw 126; a bag fixing cap on which the bag is inserted into the fixing holder 150-1 on the upper side and which is fastened with screws; a urine plastic bag 150-3 formed on the lower side to temporarily store urine; a bag guiding bag 150-4 that puts into a urine plastic bag and fixes it to the leg during walking or rehabilitation exercise; a plurality of leg fixing bands 150-5 that is connected to the upper, middle and lower ends of the bag guiding bag 150-4 and fixed with Velcro; and a body urinal position fixing band 150-6 that is connected to the uppermost end of the bag guiding bag 150-4, and attached and fixed to the Velcro attachment portion of the patient diaper pants.

Here, the bag guiding bag is configured such that in order to prevent plastic urine bags for wheelchair patients from fluttering, bursting, breaking, or leaking while the patient is walking or undergoing rehabilitation, the urine bag is placed in the bag guiding bag, and the upper end entrance is fixed by attaching a Velcro band to the patient's diaper panties in a V shape, and the elastic Velcro bands at the middle and lower parts are tightened and fixed to the thigh above the knee to allow free movement and urination.

The Velcro tape of the urinal position fixing band is attached and fixed to the patient's diaper pants, and the leg fixing band, which is a Velcro tape at the lower end, can be tightened and fixed on the thigh above the knee in the form of an elastic Velcro band.

On the other hand, the urine collection cup 120 is formed with a nipple 125 at the lower part of the drainage part 124 with a smaller diameter and protrudes downwardly to a certain length, and the nipple 125 can be connected to a straw-shaped urine drain tube 170.

On the other hand, the Velcro attachment band 115 of the body connecting and fixing device 110 may be formed to be attached and fixed to the Velcro attachment portion 201 of the patient diaper panties 180 worn by the patient.

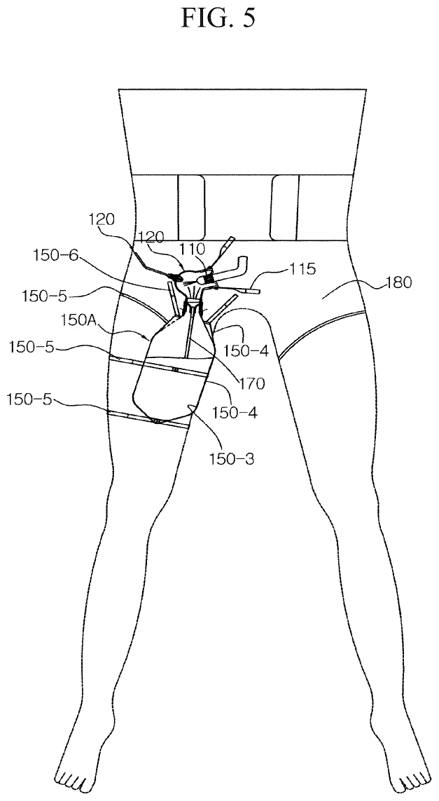

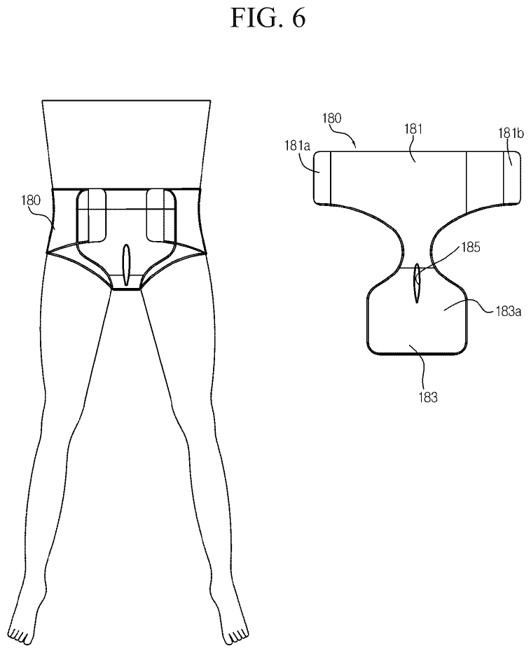

Referring to FIG. 5, the patient diaper pants 180 may comprise a back plate band 181 formed to protrude on both sides and wrap around the back plate having both left and right wings 181a and 181b having a Velcro structure; and a front plate shielding band 183 that is formed to have a constant width in the front while being connected and formed to a back plate band 181 with one connecting band, thereby covering the patient's genitals, wherein the front plate shielding band 183 is configured such that its front surface portion is composed of a Velcro attachment portion 183a, and its center is formed with a hole 185 that connects the patient's genitalia.

Here, a body-worn urinal 100 for male patients is installed around the hole 185 from which the body's genitals protrude, and the Velcro attachment band 115 of the body connecting and fixing device 110 can be tightly fixed to the Velcro attachment portion 183a of the front plate shielding band 183.

The functions and effects of the body-worn urinal for male patients of the present invention will be described as follows. The following description is based on the above structural description.

First, in the body-worn urinal 100 for male patients of the present invention, the penis of the male body is connected and worn to the body connecting fixing device 110.

Next, the body connecting fixing device 110 is detached and installed on the urine collection cup 120.

Then, while wearing the patient diaper pants 180, the patient can finish wearing the diaper by grasping both ends of the Velcro attachment band 115 that is connected and fixed to the body connecting and fixing device 110 so that it is attached and fixed to the Velcro attachment portion 183a of the patient diaper pants 180.

Even when the patient is in various pasture such as lying down during such a wearing condition, it is possible to stably perform urination through the urethra while maintaining a stable fixation state so that the connection part of the urine collection tube is rotated left and right to smoothly flow urine.

The urine released through the patient's urethra is sprayed into the water collection part 121 of the urine water collection cup 120 formed in a spherical shape in front of the glans of the genitals, and is discharged through the urine discharge hose 140 which is directly connected to the nipple 125 via the drainage part 124 along the main surface.

Urine discharged from the urine collection cup 120 through the urine discharge hose 140 is recovered in the urine container 150, and at least 2˜3 doses of urine are collected in the urine container 150.

Once the urine is collected, the end of the urine discharge hose 140 is separated from the urine container 150, and the drain stopper 155 that blocks the discharge port 154 is separated to allow urine to be discharged in a toilet or the like.

The urinal container 150 that discharges urine filled inside can be reused by washing with water.

On the other hand, the far-infrared sterilizer 130 mounted on the urine collection cup 120 can disinfect and sterilize the area around the patient's urethra once or twice a day (for 10 to 20 minutes) depending on the condition.

In addition, the diagnostic test kit 160 built into the urine container can check the health status by checking for blood in blood urine, proteinuria, diabetes, and the like with a urine test stick. Alternatively, during detailed examinations, it will be possible to provide an environmentally friendly medical device that can remove the built-in test bottle, transfer it, and send it to a pathology room.

Further, when used by intensive care patients or general patients at night, a body-worn urinal for male patients that uses a urine discharge hose in a stationary manner connects the urine discharge hose to the drainage nipple and connects it to the urine tank on the lower side to urinate. As a result, the wing structure with a rectangular incision in the center of the silicone packing of the body connecting fixing device is soft when inserted into the urethra, expands and adjusts regardless of size, and wraps around the genitals to prevent leakage. When urinating, it immediately flows into the urine collection cup, and can be used while preventing backflow.

On the other hand, body-worn urinals for general patients, wheelchair patients, or mobile male patients used during rehabilitation exercise or walking is used by providing a urine container in the form of a plastic bag, and can be used stably by using a guide bag and wearing an elastic Velcro band on the thigh of the patient wearing diaper panties.

Currently, trauma patients hospitalized for dementia, cancer, traffic accidents, and the like wear two straight urine diapers, and then a stool diaper and a cover diaper to defecate. The patients wear diapers with multiple layers of waterproof coating, which do not allow air to pass through to bee stuffy. After urination, the guardians do not know their condition, so they leave the diapers unattended, stool and urine are left unattended, causing bedsores, urethritis, cystitis, and the like, and the patient is unable to speak and suffers. To solve such problems, odor, leakage, bedsores, urethral infections, pain, and the like are eliminated by using an infrared built-in body urinal. In particular, it is possible to reduce waste generation, changing diapers 4 to 5 times a day, and fatigue of caregivers.

While the present invention has been shown and described in connection with the exemplary embodiments, it will be apparent to those skilled in the art that modifications and variations can be made without departing from the spirit and scope of the invention as defined by the appended claims.

1. A body-worn urinal for male patients comprising:
a body connecting and fixing device formed of a holder for fixing male genitalia;
a urine collection cup for collecting urine, the urine collection cup comprising a water collection part formed on the upper side thereof and having a spherical inner space, a lamp holder formed on the upper end of one side of the water collection part so as to penetrate therethrough, a connection part which is formed on the other side of the water collection part and is open such that the body connecting and fixing device is connected thereto, and a drainage part which is formed downward from the water collection part so as to discharge urine;
a far-infrared sterilizer coupled to the lamp holder of the urine collection cup;
a urine discharge hose for discharging the urine collected in the urine collection cup; and
a urine container to which the urine discharge hose is connected so as to temporarily store the urine,
wherein the body connecting and fixing device further comprises Velcro attachment bands which are connected and fixed to both side ends of the outside thereof so as to be attached and fixed to Velcro attachment portions of a patient diaper.

2. The body-worn urinal for male patients as claimed in claim 1, wherein the body connecting and fixing device comprises,
a connection coupling for which one end of the outer periphery is detachably coupled to the connecting part of the urine collection cup; and
a divided packing cap which consists of a stretchable peripheral ring separated into a plurality of numbers while protruding and extending with a certain diameter and length to the center along the inner peripheral edge of the connection coupling, and which becomes thinner in thickness toward the end, tightly adheres to the skin surface, and is stretched and adjusted regardless of size to fit closely to the genitals of the body to prevent leakage.

3. The body-worn urinal for male patients as claimed in claim 2, wherein a protruding ring part is further formed to protrude on both sides of the outer periphery of the connection coupling, and connects and fixe a Velcro attachment band.

4. The body-worn urinal for male patients as claimed in claim 1, wherein the far-infrared sterilizer comprises an infrared lamp that is coupled to a lamp holder having a hole formed at the upper end of one side of the urine collection cup, and is connected to the lamp holder by a socket and a cable.

5. The body-worn urinal for male patients as claimed in claim 1, wherein the urine container comprises a diagnostic test kit consisting of a urine test bottle and a urine test stick that are compartmentalized and formed on one side and allow urine flowing in from the urine discharge hose to be tested.

6. The body-worn urinal for male patients as claimed in claim 1, wherein the Velcro attachment band of the body connecting and fixing device is attached and fixed to the Velcro attachment portion of patient diaper pants worn by the patient.

7. The body-worn urinal for male patients as claimed in claim 1, wherein the patient diaper pants comprises,
a back plate band formed to protrude on both sides and wrap around the back plate having both left and right wings having a Velcro structure; and
a front plate shielding band that is formed to have a constant width in the front while being connected to a back plate band and one connecting band, thereby covering the patient's genitals,
wherein the front plate shielding band is configured such that its front surface portion is composed of a Velcro attachment portion, and its center is formed with a hole that connects the patient's genitalia.

8. The body-worn urinal for male patients as claimed in claim 1, comprising:
a bag fixing holder which is configured to have a connecting locking jaw formed to protrude outward with a certain width at the lower end portion of the drainage part of the urine collection cup, and which forms a urine container removably attached to the drainage part of the urine collection cup, and is coupled and fixed to the connection locking jaw;
a bag fixing cap on which the bag is inserted into the fixing holder on the upper side and which is fastened with screws;
a urine plastic bag formed on the lower side to temporarily store urine;
a bag guiding bag that puts into a urine plastic bag and fixes it to the leg during walking or rehabilitation exercise;
a plurality of leg fixing bands that is connected to the upper, middle and lower ends of the bag guiding bag and fixed with Velcro; and
a body urinal position fixing band that is connected to the uppermost end of the bag guiding bag, and attached and fixed to the Velcro attachment portion of the patient diaper pants.

9. The body-worn urinal for male patients as claimed in claim 8, wherein the drainage part of the urine collection cup has a nipple structure, which is configured to be connected to a urine plastic bag and a straw-structured urine drain pipe to drain urine.
